(19) United States
(12) Patent Application Publication
(10) Pub. No.: US 2005/0107672 A1
(43) Pub. Date: May 19, 2005

(54) System and method for external input of disease management algorithm

(75) Inventors: Randolph B. Lipscher, Austin, TX (US); Michael D. Dahlin, Austin, TX (US); Eric Wohl, Austin, TX (US)

Correspondence Address: TOLER & LARSON & ABEL L.L.P., 5000 PLAZA ON THE LAKE STE 265, AUSTIN, TX, 78746, US

(73) Assignee: RECARE, Inc.

(21) Appl. No.: 10/959,912

(22) Filed: Oct. 6, 2004

Related U.S. Application Data
(63) Continuation-in-part of application No. 09/992,035, filed on Nov. 23, 2001.
(60) Provisional application No. 60/508,998, filed on Oct. 6, 2003.
(60) Provisional application No. 60/252,820, filed on Nov. 22, 2000.

(51) Int. Cl. A61B 5/00

(52) U.S. Cl. 600/300

(57) ABSTRACT

The disclosure is directed to a system including a disease management advisor system configured to receive a disease management algorithm from a disease management advisor. The system further includes a healthcare provider system communicatively coupled to the disease management advisor system and configured to receive the disease management algorithm from the disease management advisor system. The system also includes a conversion module accessible to the healthcare provider system and configured to convert the disease management algorithm based on a controlled medical vocabulary.

### CROSS-REFERENCE TO RELATED APPLICATION(S)

The present application claims priority from U.S. Provisional Application No. 60/508,998, filed Oct. 6, 2003, entitled “SYSTEM FOR EXTERNAL INPUT OF DISEASE MANAGEMENT ALGORITHM AND INTEGRATION OF ALGORITHM AT POINT OF CARE,” naming inventors Randolph B. Lipscher and Michael D. Dahlin, which application is incorporated by reference herein in its entirety.

The present application is a continuation-in-part of and claims priority from U.S. patent application Ser. No. 09/992,035, filed Nov. 23, 2001, entitled “SYSTEM AND METHOD FOR INTEGRATING DISEASE MANAGEMENT INTO PHYSICIAN WORKFLOW,” naming inventors Michael D. Dahlin, Eric Wohl, Randolph B. Lipscher, and Charles Andrew Bergman, which claims priority to U.S. Provisional Application No. 60/252,820, filed Nov. 22, 2000, entitled “SYSTEM AND METHOD FOR INTEGRATING DISEASE MANAGEMENT INTO PHYSICIAN WORKFLOW,” both of which applications are incorporated by reference herein in its entirety.

### FIELD OF THE DISCLOSURE

This disclosure, in general, relates to systems for external input of disease management algorithms and integration of algorithms at points of care.

### BACKGROUND

Healthcare costs are increasing and, as a result, payers, such as government agencies and insurance companies, are seeking to reduce costs. Government agencies, such as Medicaid and Medicare, are experiencing large increases in expenditures. Insurance companies are also experiencing cost pressures and are increasing policy rates.

In many cases, payers have implemented rules and conditions under which they will pay for a procedure or examination. In addition, these payers have developed disease management programs to manage disease treatment and prevent or mitigate expensive consequences to manageable diseases, such emergency room visits and hospitalization.

However, insurance companies, government agencies, and other organizations associated with health services have difficulty communicating with physicians. Physicians generally have greater access to the patient. When physicians lack information about a payers rule or a specific patients disease management program, they may not conform to the rules or guide the patient's compliance with the disease management program. As such, an improved system for providing input of disease management algorithms to disease management systems and integrating the algorithms into physician workflow would be desirable.

### SUMMARY

In one particular embodiment, the disclosure is directed to a system including a disease management advisor system configured to receive a disease management algorithm from a disease management advisor. The system further includes a healthcare provider system communicatively coupled to the disease management advisor system and configured to receive the disease management algorithm from the disease management advisor system. The system also includes a conversion module accessible to the healthcare provider system and configured to convert the disease management algorithm based on a controlled medical vocabulary.

In another embodiment, the disclosure is directed to a disease management system including an input interface configured to receive a disease management algorithm, a selection module configured to select a set of recipients for the disease management algorithm, and a transmission module configured to transmit the disease management algorithm to a healthcare provider system associated with at least one recipient of the set of recipients.

In a further exemplary embodiment, the disclosure is directed to a healthcare provider system including an algorithm reception interface for receiving a disease management algorithm, storage responsive to the algorithm reception interface and configured to store the disease management algorithm, and a medical data module configured to provide a medical data based on the disease management interface.

In another exemplary embodiment, the disclosure is directed to a method of disease management. The method includes receiving a disease management algorithm from a disease management advisor, mapping the disease management algorithm to a controlled medical vocabulary, and providing a medical data entry interface based oil the controlled medical vocabulary. The medical data entry interface is configured based on the disease management algorithm.

### BRIEF DESCRIPTION OF THE DRAWINGS

The present disclosure may be better understood, and its numerous features and advantages made apparent to those skilled in the art by referencing the accompanying drawings.

FIGS. 1, 2 and 3 include diagrams illustrating exemplary disease management systems.

FIGS. 4 and 5 include flow diagrams illustrating exemplary methods for using disease management systems, such as those illustrated in FIGS. 1, 2, and 3.

FIGS. 6 and 7 include diagrams illustrating exemplary disease management advisor systems.

FIGS. 8, 9, 10, 11, and 12, include flow diagrams illustrating exemplary methods for use by disease management systems, such as those illustrated in FIGS. 1, 2, and 3.

FIGS. 13, 14, and 15 include general diagrams illustrating exemplary interfaces for use in a medical workflow.

FIG. 16 includes a flow diagram illustrating an exemplary method for use by a disease management system, such as those illustrated in FIGS. 1, 2, and 3.

FIG. 17 includes a flow diagram illustrating an exemplary medical workflow.

FIGS. 18-29 include general diagrams illustrating exemplary interfaces for use in a medical workflow.

The use of the same reference symbols in different drawings indicates similar or identical items.

### DESCRIPTION OF THE PREFERRED EMBODIMENT(S)

In one particular embodiment, the disclosure is directed to a system including a disease management advisor system configured to communicate with a healthcare provider system. The disease management advisor system transmits a disease management algorithm to the healthcare provider system. The disease management algorithm is converted or mapped to a controlled medical vocabulary for use by the healthcare provider system in providing an interface for entry of medical information. For example, the interface may include control elements for entering patient medical information. In one particular embodiment, the interface is configured based on the disease management algorithm.

In one exemplary embodiment, the disease management algorithm includes a set of rules that determine when the algorithm is applicable and includes data associated with the action resulting from application of the disease management algorithm. The disease management algorithm may also include information regard in which HCP systems it should be implemented. For example, a formulary disease management algorithm may include rules that direct implementation of the algorithm when a specific drug is prescribed or when a prescription interface is displayed in conjunction with a patient examination that reveals a particular set of findings. Such a formulary algorithm may be implemented in all HCP systems associated with a particular disease management advisor. In another exemplary embodiment, a patient-based disease management algorithm may be implemented to display a particular text message when a particular patient visits a physician. In such an example, the algorithm would be sent only to HCP systems associated with the treatment of the particular patient. In general, the disease management algorithm is interpreted to configure interfaces provided by the HCP system.

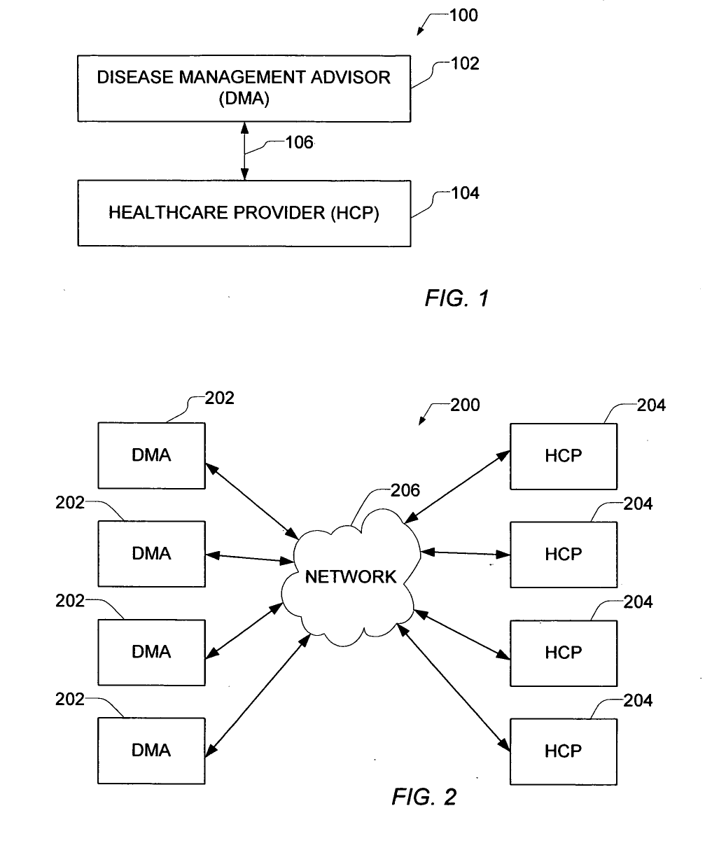

FIG. 1 shows the top-level system architecture. The system 100 comprises at least one Disease Management Advisor (DMA) system 102, at least one Health Care Practitioner (HCP) system 104, and at least one network 106 connecting these systems.

A disease management advisor system 102 is a system used by one or more disease management advisors. A disease management advisor (DMA) includes an entity that advises one or more health care providers regarding treatment of patients. Examples of disease management advisors include specialized disease management advising companies, pharmacies, insurance companies, government agencies, medical professional societies, medical specialists, experts in particular fields of medicine, pharmacy benefits management companies, pharmacy advertisers, pharmacies, labs, research institutions, organizers of medical studies, and organizers of medication trials.

A health care practitioner system 104 or a healthcare provider system is a system used by one or more health care practitioners. A health care practitioner (HCP) is an entity that participates in the examination, evaluation, or treatment of medical patients. Examples of health care practitioners include medical doctors, nurses, nurse practitioners, clinic administrative staff, technicians, pharmacologists, pharmacy workers, medical students, and medical assistants.

In some exemplary embodiment, the “HCP system” and “DMA system” roles may both be played by the same entity at different times. Generally, an HCP is an entity that executes examination, evaluation, or treatment of a patient. Generally, a DMA is an entity that advises about health care decisions. Often, a doctor acts as an HCP and an HMO acts as a DMA. In alternative embodiments, an HMO acts as the HCP (e.g., approving a treatment) while a doctor acts as a DMA (e.g., recommending a treatment). In some exemplary embodiments, a patient acts as an HCP (e.g., administering a medication) while a doctor acts as a DMA (e.g., providing information about a medication's side effects.) In a further example, a patient acts as a DMA (e.g., requesting a medication refill) and the doctor acts as an HCP (e.g., ordering a medication refill).

In an exemplary embodiment, the computer network 106 includes the Internet and DMA systems and HCP systems are physically separate computer systems. In another exemplary embodiment, the computer network 106 is a shared memory system through which DMA and HCP systems resident in the same physical computing system communicate.

In an exemplary embodiment, the computer network 106 includes an encryption modules and the system uses the encryption modules to authenticate communication and to prevent third parties from being able to read information. In this embodiment, authentication ensures that DMA systems and HCP systems know the identity of the systems with which they communicate or the identity of the users of those systems, and the DMA and HCP system restrict access to system functionality based on the identity of the system or user requesting an action. Encryption may be accomplished using algorithms known to practitioners skilled in the art, such as DES, IDEA, Blowfish, and RSA. Authentication may be accomplished using algorithms known to practitioners skilled in the art, such as Kerberos, PGP, public key cryptography, SDSI, and SPKI.

In a particular embodiment, each DMA system 202 communicates with multiple HCP systems 204 and each HCP system 204 communicates with multiple DMA systems 202 through a network 206, as illustrated in FIG. 2. Each of the DMA systems 202 and HCP systems 204 may include a transmission module that permits addressing and transmission of data and disease management algorithms between systems. In an alternative embodiment, a central transmission system may receive the disease management algorithms from each of the systems and determine to which system the disease management algorithm is to be transferred.

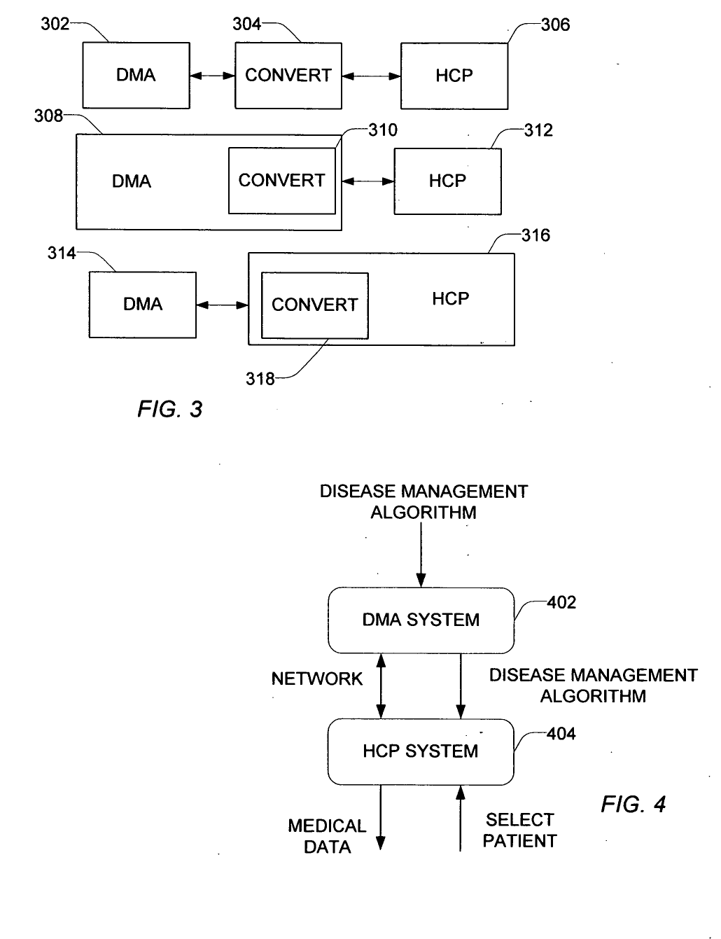

As FIG. 3 illustrates, the system may also include a conversion module, such as conversion modules 304, 310, and 318. Typically, the HCP system, such as HCP systems 306, 312, and 316, will manipulate and store medical data that describes medical conditions using a controlled medical vocabulary. A controlled medical vocabulary defines a unique word or identifier to each unique significant medical concept. Synonyms or other phrases describing the same concept are mapped to the controlled medical vocabulary's identifier. For example, a controlled medical vocabulary might map “elevated temperature”, “feels warm”, and “fever” to the term “fever” or, in another embodiment, to unique number, such as 284751. Example controlled medical vocabularies include SNOMED-CT (Systematized Nomenclature of Medicine—Clinical Terms), ICD9-CM, LOINC (Logical Observation Identifiers Names and Codes), READ, and UMLS (Unified Medical Language System). In an embodiment, one or more DMA systems, such as DMA systems 302, 308, and 314, may use different controlled medical vocabularies from the controlled medical vocabulary used by the HCP system, such as HCP systems 306, 312, and 316. In addition, the HCP systems (e.g. 306, 312, and 316) may use different controlled medical vocabularies from each other. In this embodiment, a conversion module (i.e. 304, 310, and 318) translates rules specified at a DMA system using the DMA's controlled medical vocabulary to rules executed at the HCP system using the HCP's controlled medical vocabulary. In one embodiment, this conversion module includes a mapping from each term in a DMA's controlled medical vocabulary to a mapping in the HCP's controlled medical vocabulary and translates each instance of a term from the former into a term from the latter.

In one embodiment, a DMA system may specify rules without using a controlled medical vocabulary. In such an embodiment, the conversion module translates from the set of terms or phrases that refer to a concept to the concept in the HCP's controlled medical vocabulary. In one embodiment, this translation comprises a human expert reading the DMA rule and converting it to a rule in the HCP system's controlled medical vocabulary. In another embodiment, this translation is done automatically using, for example, the method described by Kornai and Stone (Andras Kornai and Lisa Stone, “Automatic Translation to Controlled Medical Vocabularies” in A. Abraham and L. Jain (eds): Innovations in Intelligent Systems and Applications Springer Verlag, 2004, 413-434), included herein by reference.

As FIG. 3 illustrates, the conversion occurs between arrival of data at the DMA system and display of data at the HCP system, but the system may be constructed with this conversion occurring at various places. For example, this conversion may occur after the algorithms have been sent by the DMA and before they are received by the HCP (e.g., conversion may occur at a data center 304 through which the DMAs and HCPs communicate.) Alternatively, this conversion may occur within the DMA system, as illustrated by DMA system 308 and conversion module 310. In another example, this conversion may occur within the HCP system, such as HCP system 316 having conversion module 318.

FIG. 4 depicts four data flows in the system. The disease management advisor system 402 receives one or more disease management algorithms and, in one embodiment, stores the algorithms. The disease management advisor system transmits the one or more disease management algorithms across the network to one or more health care practitioner systems 404, which store the one or more algorithms. The health care practitioner system 404 receives medical inputs including a directive to select a patient. The health care practitioner system displays medical data, the contents of which depend upon both the patient selected and at least one of the disease management algorithms received.

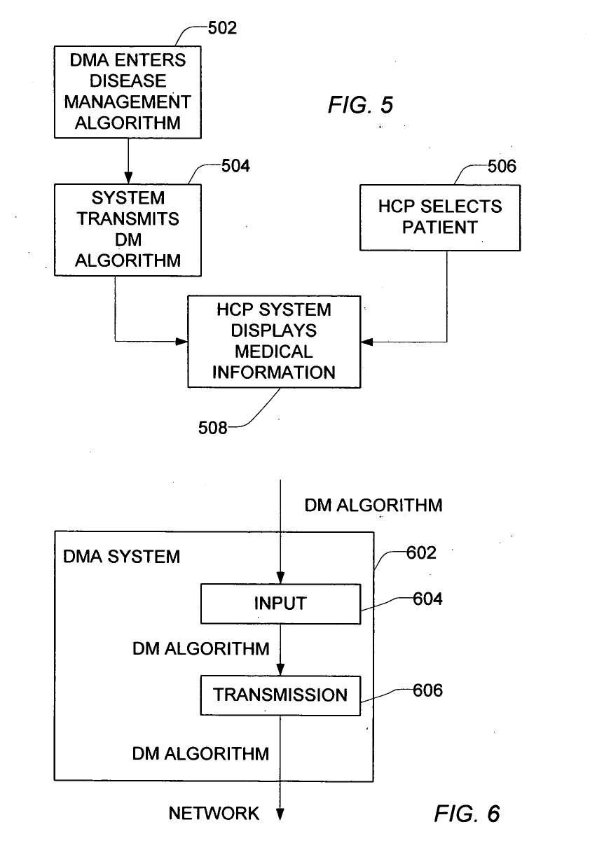

FIG. 5 indicates a flow chart of actions relating to these flows of data. A DMA enters at least one disease management algorithm into a DMA system, as shown at step 502. After the entry of the at least one disease management algorithm, the network transmits the at least one disease management algorithm from a DMA system to an HCP system, as shown at step 504. An HCP selects a patient at the HCP system, as shown at step 506. After the HCP selects a patient, the HCP system displays medical data specified by the disease management algorithm for the selected patient based on the algorithm's operation on medical data associated with the selected patient, as shown at step 508. Note that the selection of the patient may precede or follow the network transmission of the at least one algorithm.

One can envision various methods by which an algorithm can analyze the records associated with a selected patient in order to specify medical information to be displayed. In one embodiment the disease management algorithm specifies one or more selection conditions and specifies medical data. The HCP system displays an algorithm's medical data when the selection conditions specified in the algorithm are satisfied by the record associated with the selected patient.

Medical data include data relating to medical treatment. Examples include findings, medical reference information, medical journal articles, best practice recommendations or guidelines, alerts about a patient, information about a medical condition, information about a doctor, recommended questions, recommended actions, forms, approval of medical orders, medications or tests, orders for medications, treatments or tests, medical coding information, changes to existing templates or forms, and the like. A finding is information about a particular medical patient. Examples of such information include: demographic information (such as the patient's age, gender, address, or occupation), symptom information, disease process information, medical history information, family history information, behavioral information (such as patient alcohol consumption or smoking status), medication, genetic information, allergy information, reaction information, current medication or treatment information, past medication or treatment information, payer information (such as insurance company or pharmacy benefits management company), and other observations of or information about a patient. Note that findings are a particular category of medical data.

A selection condition specifies conditions for which medical data should be displayed. These conditions are based on one or more factors. In one embodiment, these factors include at least one medical finding about the currently selected patient. In another embodiment, the selection condition also includes at least one factor regarding other information about the selected patient (e.g., the patient's payer or home address), information about the HCP user (e.g., the user's specialty, identity), or the current task being performed by the HCP system (e.g., “displaying Review of Systems template”, “prescribing medication”, “ordering MRI”). A selection condition includes a function taking factors as input. Example functions include boolean functions, a numerical function, numerical range comparisons, database queries, string regular expressions, computer program functions, and combinations of these types.

To illustrate examples of selection conditions: a selection condition may specify that medical data be displayed (1) when a particular HCP selects a particular patient and begins the “view current messages” task, (2) when any HCP selects any patient that has a particular condition, (3) when any HCP selects any patient enrolled in a particular insurance plan, or (4) when any HCP selects any patient enrolled in a particular insurance plan and who has a particular condition and the current HCP task associated with the patient is the “prescribe medication” task.

In one embodiment, each disease management algorithm specifies one or more selection conditions and specifies at least one from a list of standard system response actions. The HCP system activates the response action when the selection conditions specified in the algorithm are satisfied by the record associated with the selected patient. In one embodiment, one or more of the disease management algorithms also includes medical findings. In this embodiment the HCP system activates the response action using the medical findings as arguments or modifiers to the response action when the selection conditions specified in the algorithm are satisfied by the record associated with the selected patient.

System response actions are “hooks” in the HCP system for altering or augmenting the behavior of the HCP system. By specifying the disease management algorithms in terms of a relatively short list of standard actions, a general and extensible framework can be constructed that allows a broad range of different DMA entities to specify a broad range of modifications to the base HCP system.

For example, the standard system response actions may include at least one of:

(1) Prompt the user for more information. An algorithm that includes this action also specifies a medical findings template that is to be displayed. This template provides input elements (e.g., checkboxes, tri-state boxes that can be marked “positive”, “negative” or “unmarked”, text input fields, etc.) with each entry tied to a specific finding in the HCP system's controlled medical vocabulary. When such a template is displayed, the state of the inputs is initialized according to the current patient's electronic medical record as stored in the HCP system (e.g., if the user checked the box for “difficulty swallowing” on the Physical Exam page and then the response action displays a template that includes a “difficulty swallowing” field, the field would appear in the “checked” state). Also, when updates are made to the displayed template, these updates are propagated back to the “standard” HCP template display (so that if the user unchecks “difficulty swallowing” in the displayed template and then views the standard Physical Exam template, the “difficulty swallowing” box will now appear unchecked.

(2) Send a message to an external entity. An algorithm that includes this action also specifies one or more medical findings from the patient's record to be sent and a destination to which this information should be sent. For example, an algorithm might specify that whenever a patient insured by Blue Cross is diagnosed with diabetes, that patient's name, contact information, diagnosis, chief complaint, history of present illness, review of systems, physical exam, current medication, and past medical family and social history be sent to a disease management nurse who will contact that patient to coordinate that patient's care. Destinations are specified using addresses, such as email addresses or DMA system specific addresses, or user IDs from the HCP and DMA systems. Note that confidentiality of medical information is important. Embodiments may protect confidentiality in two ways. First, before any information is sent, the user (typically the physician treating the patient) is notified of the destination and the information being sent, and the user is prompted to approve transmission before it is sent (or decline transmission to cancel it.) Second, the system includes a set of rules restricting transmission of information about a patient to specific destinations. For example, in a given HCP system, transmission might be permitted to healthcare workers in the clinic, specialists allied with the clinic or the patient's plan, the patient him/herself, the patient's guardian, or the patient's payer.

In another embodiment, transmission to researchers and government agencies is supported by stripping any personally identifying information from the list of findings to be transmitted for algorithms that specify such an entity as the recipient. Additionally, such transmission may pass through a delay aggregator that collects a number of records and or delays transmission of such records in time before sending them to the destination.

(3) Display information. An algorithm that includes this action also specifies the information to be displayed. For example, this information could include a recommendation for best-practice treatment (e.g., “Antibiotic X is more effective than antibiotic Y for condition Z”), an advertisement, prescribing information, summaries of recent related clinical studies, a warning (e.g., “medication X is contra-indicated for the current patient because the patient has finding Y”, or “Mr. Jones [the current patient] is allergic to medication class Z”) or opportunities to enroll patients in medical trials. This information can include text, graphics, and references such as URLs that can be clicked on to display additional information.

(4) Display a dialog. A dialog includes information of the types listed above for “display information”. However, the dialog response may solicit explicit action by the user (e.g., click “ok”) in order to force the user to take some action (at a minimum to acknowledge) the information.

(5) Payer approval. This action specifies a list of findings and a set of rules across those findings that must be satisfied for automatic approval of a procedure, medication, or order. When a payer approval action is triggered, the system displays the findings (along with their current states from the patient's record) and allows the user to update these findings on the on-screen template. If the state of the findings satisfies the criteria, a message to that effect is displayed. The user can select “done,” for example, if the treatment is automatically approved or if the user chooses not to proceed with the approval process. Alternatively, the user can select “request override”. The action specifies an approval agency and a set of findings to be sent to the approval agency, and when the user requests an override, this information is displayed and the user also may (1) enter (in text or via voice dictation) additional comments and (2) select additional parts of the patient's record (e.g., the physical exam, review of systems, etc.) to include in the transmission. When the user selects “send,” the information is sent to the approving entity.

In one embodiment, the transmission includes a capability allowing the approving agency to transmit electronically an approval number that the HCP system binds to the current patient's current encounter record. In another embodiment, the approving agency inserts a new algorithm into the system specifying that a message indicating approval be displayed when the current user selects the current patient and either views the current encounter record or views the “messages” task.

(6) Highlight. This action specifies a particular finding that appears on the screen that is currently being displayed by the HCP system when the algorithm is triggered. The HCP system realizes this action by highlighting on screen the specified finding. This action can, for example, allow a pharmacy benefit management company to highlight a medication on a list of medications because that medication is both on-formulary for the patient and conforms to best-practice guidelines for the patient's condition.

(7) Preselect. This action specifies that a particular finding that appears on the screen be set to the “selected” state. For example, the algorithm specifies that for a patient with condition X, a blood test of type Y be ordered if no order for test Y appears in the patient's record for the past year. In one embodiment, algorithms of this type are restricted to (a) only work on findings that correspond to orders (e.g., not physical exam findings or HCP findings) and (b) only findings that appear on the current screen (e.g., the algorithm can not change the state of a finding that is “invisible” to the user.)

It will be apparent to those skilled in the art that other system responses can be defined. For example, a system response that combines attributes from the above responses could be defined.

Referring back to FIG. 3, in one embodiment, in addition to converting rules between vocabularies, the conversion module converts a DMA's rules from a list or table of criteria to the system's disease management algorithm format. For example, the United States Government currently publishes tables of criteria that must be met in order for specific medical procedures, orders, medications, or billing codes to be authorized. For example, such a table may specify that Hospice Care may be ordered (and paid for by a specific government program) when the patient is expected to live fewer than 6 months. The conversion module maps this table of rules to specific conditions (e.g., a finding that corresponds to one or more controlled medical vocabulary elements corresponding to “patient not expected to survive 6 months”—e.g., a single code for that concept, or any of a group of codes including specific diagnoses where the standard prognoses is that the patient will die within 6 months if the disease runs its expected course.) and to specific actions (e.g., when “hospice care” is selected as an order and the condition is not met, display a warning to the user.)

In one embodiment, the system (1) downloads a set of rules, (2) converts these rules to one or more disease management algorithms specifying (a) at least one selection rule operating on a medical finding element specified by a controlled medical vocabulary identifier and (b) at least one action to be taken, (3) receives a patient selection from a user, (4) evaluates the selected patient's medical record under the selection rule, and (5) depending on the result of the evaluation of the selection rule, takes the specified action or takes some other action. (Note that the latter case includes taking no action.) In one embodiment, the rules downloaded from the public Internet are specified using a different controlled medical vocabulary than the selection rules used internally by the system.

As illustrated in FIG. 6, in an exemplary embodiment, the disease management advisor system 602 provides an interface 604 for input of one or more disease management algorithms and an interface or module 606 for transmission of these one or more algorithms across a network to one or more HCP systems. The DMA system receives as input one or more disease management algorithms via the input interface 604 and sends one or more disease management algorithms to one or more HCP systems using a network via the transmission module 606.

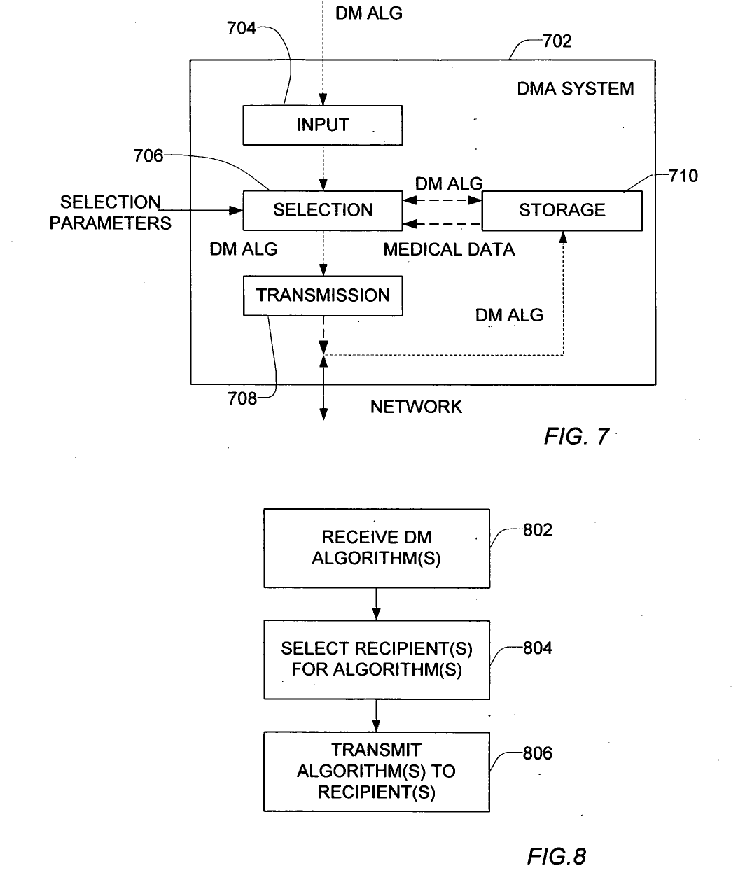

The algorithms may be sent to one HCP system, a subset of HCP systems, or all HCP systems. In one embodiment, illustrated in FIG. 7, the DMA system also includes a selection module 706 that chooses to which HCP systems a particular algorithm should be sent.

In this embodiment, the selection module 706 makes use of a storage module 710, such as DRAM memory, disk, a database, or a file system. In one embodiment, the storage module 710 stores information relevant to the selection conditions in one or more algorithms. For example, in one embodiment, the storage module 710 stores information about patients, such as which patients are treated by which HCPs or such as patient findings. For example, in one embodiment, the storage module 710 stores the current task being performed by an HCP using a particular device, which allows relevant disease management algorithms to be transmitted to an HCP system while the HCP system is being used to treat a particular patient. In another embodiment, the storage module 710 stores information about a particular clinic or user that uses the HCP system. For example, in one embodiment the storage module 710 stores information about HCP devices, such as which HCP devices are used by which HCPs or which insurance plans an HCP user is counted as a primary care physician.

In this embodiment, the system acts as follows. The DMA system 702 receives as input one or more disease management algorithms via an input interface 704 and receives as input one or more selection criteria and sends one or more disease management algorithms to one or more HCP systems using a network connected to transmission module 708. In this embodiment, the set of disease management algorithms sent to a given HCP system depends on applying the selection criteria to characteristics of the HCP system or medical information about one or more patients served by the HCP system or on both.

FIG. 8 illustrates an exemplary flow of actions performed by an exemplary DMA system. The system receives one or more input algorithms, as shown at step 802, selects destinations to which these algorithms are sent, as shown at step 804, and transmits these algorithms, as shown at step 806.

In one embodiment, the storage module used by the selection module receives medical data from the HCP device. For example, the HCP device may use a network to transmit the list of HCPs registered to access the device, the list of patients treated by users of the device, and findings about patients. The DMA system receives and stores this information.

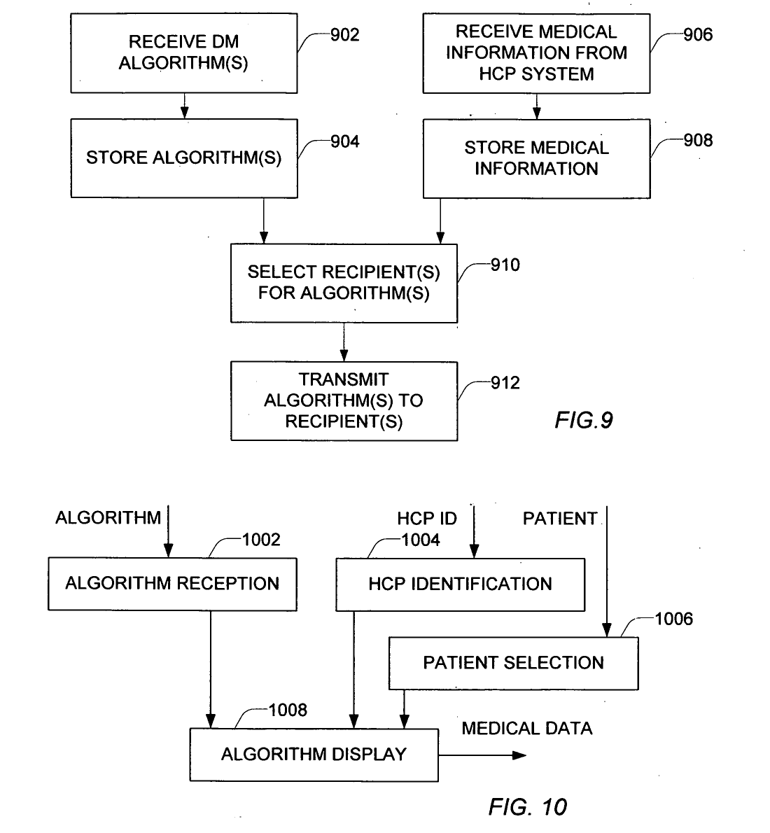

FIG. 9 illustrates an exemplary flow of actions performed by an exemplary DMA system. The system receives and stores one or more input algorithms, as shown at steps 902 and 904, and the system receives and stores one or more medical data elements, as shown at steps 906 and 908. The system selects recipients for algorithms based on selection conditions in the algorithm applied to stored medical data, as shown at step 910. The system then sends the algorithms to the selected recipients, as shown at step 912. Note that in this exemplary embodiment, selection and transmission of algorithms may be triggered by arrival of either an algorithm or of medical data. For example, the system may receive one algorithm, and transmit that algorithm whenever it receives medical data indicating that a new patient matches the selection criteria for that algorithm.

The input interface provides algorithms to the DMA system. For example, the input interface may be a keyboard, mouse, touch screen, graphic user interface, another computer, another program or stored information, among others.

The data entered via the input interface specifies one or more disease management algorithms. In an exemplary embodiment, the input interface includes a method to input one or more selection condition specifiers, such as the identity of an HCP recipient, the identity of a patient, a set of findings to match, and/or a task performed by the HCP using the HCP system. In an exemplary embodiment, the input method also includes a method to input one or more medical data items, such as a message, form to be filled out, alert to be displayed, and/or modification to be made to an examination template. The input method processes the data received and generates an algorithm data structure in a machine-readable form such as XML, an in-memory data object, HTML, formatted text, a database record, or the like.

In one exemplary embodiment, the input method includes a Graphical User Interface (GUI) display modeled on a typical HTML-based web-based email service. However, where a typical HTML-based web-based email interface provides a “To:” field in which the email address of a user is specified, the Input GUI of the exemplary disease management system includes a field for specifying a patient ID and one for specifying an HCP ID. In one particular embodiment, these fields may include the option to specify that a disease management algorithm apply to all patients or all HCPs. Where a typical HTML-based web-based email interface provides a text input area, the GUI also includes an area for entering a text message. The interface may alternatively include a field for entering selection criteria. Where a typical HTML-based web-based email interface provides a button to “send” the message, the input elements provides a button to “send” the algorithm.

In this exemplary embodiment, the user enters a patient ID, HCP ID, and message and activates the send button. The input method processes the field inputs to assemble an XML record in which the patient ID and HCP ID are selection condition items indicating that the algorithm pertains to the patient P and HCP H specified in the corresponding GUI fields and in which the text message M from the text GUI field is included as medical data. In one exemplary embodiment, this algorithm is interpreted to mean “when HCP H selects patient P, display message M”.

In a second exemplary embodiment, the input method includes a text editor such as emacs, Microsoft Notepad, or vi with which a user specifies algorithms as XML data files. Input is accomplished by signaling the disease management advisor system to load one or more specified data files from local storage or from across a network.

The transmission module transmits algorithms to one or more HCP systems. In one embodiment, all algorithms are transmitted to all HCP systems. In another embodiment, algorithms to be transmitted include a list of HCP systems to which they are to be transmitted. In one embodiment, transmission is immediate. In another embodiment, the transmission module stores the messages to be transmitted and transmits them at a later time.

The transmission module uses a network protocol to deliver data to the remote HCP system. Algorithms embodying such protocols are known to those skilled in the art, such as HTTP, TCP, RPC, and shared memory. In an exemplary embodiment, the transmission is accomplished using a protocol that encrypts transmitted data such as SSH, SSL, or DES over TCP. Although the system can be implemented by transmitting all algorithms to all HCPs or by requiring the DMA user to explicitly specify specific HCPs that should receive specific messages (as illustrated in FIG. 6), an exemplary embodiment (illustrated in FIG. 7) also includes a selection module that selects which HCP systems should receive each algorithm.

In one embodiment, the algorithms are be transmitted soon after they are received by the DMA system. In this embodiment, the system receives a disease management algorithm, selects a set of HCP systems to which the algorithm should be sent by comparing the algorithm's selection conditions to the stored information. The system then sends the algorithms to the selected HCP systems.

For example, the system could receive an algorithm specifying that a specified message be displayed when any HCP is treating a patient that is enrolled with a particular insurance company and that is diagnosed with diabetes. In this case, the exemplary selection module sends the algorithm to all HCP systems.

In another example, the system can receive an algorithm specifying that all HCP's treating a particular patient should be informed that that patient has recently missed an appointment for diabetes management program meeting. The exemplary selection module reads from the storage module to determine the list of HCP systems used by HCPs that treat the specified patient and sends that algorithm to that list of HCP systems.

In a further example, the system can receive an algorithm specifying a message containing the results of a test on a particular patient that was ordered by a particular HCP. The exemplary selection module sends the algorithm to the HCP system used by that HCP.

In another embodiment, the algorithms are stored and transmitted at a later time. When the algorithms are stored and transmitted at a later time, factors that select when an algorithm is transmitted include factors such as time of day, elapsed time since the algorithm was stored, receiving a request from an HCP system, receiving medical data that trigger a match with the algorithm. For example, in order to prevent data transfers from interfering with other system activities, the DMA system may store algorithms to be sent to an HCP and deliver them in a batch (a) at a specified batch update time such as 1 AM or (b) when the HCP system contacts the DMA system and requests that stored algorithms be sent.

For example, the DMA system may send the algorithms relating to patient P when triggered by a notification from an HCP system that the user of the HCP system has selected patient P as the current patient. In another example, the DMA system may send the algorithms relating to patient P when triggered by a notification from an HCP system that patient P has an upcoming appointment with clinic served by the HCP system. In a further example, the DMA system may send the algorithms relating to patient P and task T when triggered by medical information from an HCP system that the user of the HCP system has selected patient P as the current patient and has selected task T. For example, the DMA system may send algorithms relating to a patient's medication formulary when the HCP system begins the task of writing a prescription for the patient.

In a further example, the DMA system may send the algorithms relating to one or more findings criteria F when triggered by medical information from an HCP system that findings stored for a patient match criteria F. For example, when the HCP system sends medical data indicating that the finding “diagnosis=diabetes” has been entered for a patient, the DMA system may send algorithms that display information on how to enroll a patient for a diabetes management program.

In another example, when previously stored findings indicate that a patient is demographically eligible for a particular drug trial and a new finding is received from an HCP system and stored, and this new finding is a diagnosis that matches the selection diagnosis for the medical trial, the DMA system sends an algorithm alerting the HCP that the patient is eligible for enrollment in the specified medication trial.

Selection may occur at the DMA system or at the HCP system. One or both may store the needed information and may perform selection operations. Selection tasks may be partitioned to one or both in various combinations. In one example, all algorithms may be sent to all HCPs and filtered at the HCP. In another example, all data and events may be sent to the DMA and filtering performed at the DMA. In a further embodiment, filtering and selection tasks may be shared between the HCPs and DMAs.

The DMA system can be a PC, server, multiprocessor, distributed collection of machines connected by a network, a server with separate access terminals that communicate with the server with some suitable protocol (e.g., X, HTTP, VNC, tty “dumb” terminal).

In one example, the DMA system may include a CPU and memory. The DMA system may further include an input/display: screen, keyboard, and/or mouse. The DMA system may further include persistent storage and may include a network interface. The DMA system may also communicate using encrypted communication. For example, the DMA may utilize authentication and privacy features. In one exemplary embodiment, the DMA may only accept messages from the HCP. In another exemplary embodiment, the HCP may only accept messages from the DMA.

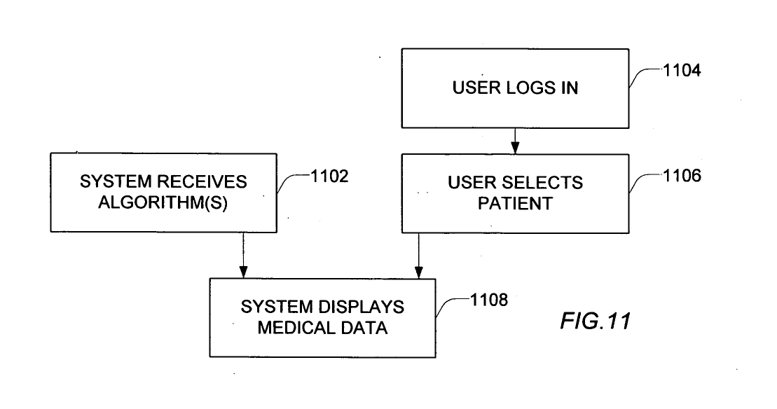

FIG. 10 illustrates components of an exemplary embodiment of the HCP system. The embodiment includes a reception module 1002, an HCP identification module 1004, a patient selection module 1006, and an algorithm display module 1008. In this embodiment, as illustrated in FIG. 11, the HCP system receives one or more algorithms from the network, as shown at step 1102. A user logs in (thereby specifying to the system the HCP ID of the user), as shown at step 1104. The user specifies a patient to examine or treat, as shown at step 1106. The algorithm display module 1004 displays medical data from one or more algorithms, as shown at step 1108.

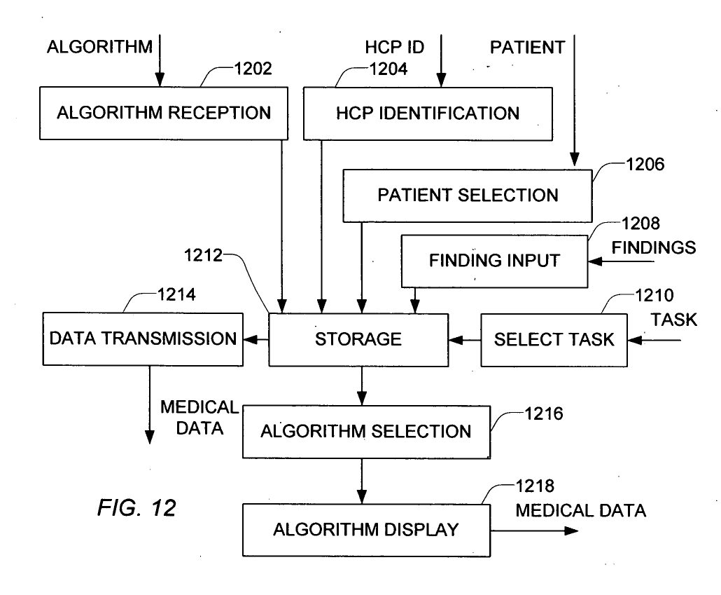

FIG. 12 illustrates components of an exemplary embodiment of the HCP system. The exemplary embodiment includes an algorithm reception module 1202, an HCP identification module 1204, a patient selection module 1206, a finding input module 1208, a task selection module 1210, storage 1212, an algorithm selection module 1216, and an algorithm display module 1218. This embodiment also includes a medical data transmission module 1214.

In this embodiment, the system receives and stores one or more algorithms. A user is identified, selects a patient, selects a task, and enters zero or more findings about the patient. The algorithm selection system 1216 selects one or more algorithms to display by matching algorithm conditions against the HCP ID, patient ID, current task, and/or findings about the patient. Note that the findings may have been entered during the current session or retrieved from storage from data entered into or loaded into the system at a previous time. The algorithm display module 1218 then displays medical data associated with the selected algorithm(s) to the user.

In this embodiment, the selection and display functions are triggered each time a different user logs in, each time a user selects a different patient, each time a user activates a task, and each time a user adds or updates a finding. In another embodiment, the selection and display functions are triggered by a subset of these events. In this embodiment, stored medical data such as the current logged-in HCP, the current patient, findings, or the current task may also transmitted to one or more DMA systems. In another embodiment, a user other than the user treating the patient selects the patient. For example, a receptionist selects a patient for the HCP to treat.

The HCP identification module 1204 allows a user to signify the identity of the user currently using the HCP system. In a particular embodiment, the HCP identification module 1204 is a log-in module in which the user enters a user ID and password. The system verifies that the entered password matches the stored password for the specified user ID, and, if so, stores the fact that the specified user is the current user of the HCP system. In another embodiment, the HCP identification module 1204 associates an HCP system with a specific user.

The patient selection module 1206 selects a patient that an HCP will treat or examine while using the HCP system. In a particular embodiment, the current user of the HCP system selects a patient from a list of patients stored in and displayed by the HCP system. At least two views of this list are supported. First, the user may view and select from all patients that are customers of the HCP's clinic, department, practice, or hospital. Second, the user may view and select from those patients that have checked in with the receptionist. In another embodiment, a user other than the current user selects a patient for the current user to treat or examine. For example, a nurse or receptionist may select the next patient that a doctor should treat.

In another embodiment, the current patient on an HCP device is selected based on the room in which that HCP device is located. In this embodiment, a receptionist enters the patient ID of the patient in a specific room and indicates the room in which that patient is located. If the HCP system is a portable device, when the HCP carries his or her HCP system into the room, the HCP system determines the room it is in using a location method such as Cricket, a bar code reader, or an IR Transponder. The patient in that room becomes the selected patient for the HCP system. If the HCP system is a lion-portable device with different HCP systems in different rooms, the assignment of a patient to a room automatically makes the specified patient the selected patient for the HCP system.

In a further embodiment, the current patient on an HCP device is selected based on patient input. In this embodiment, a patient logs into the HCP device using a smart card and becomes the current patient of the HCP device. Optionally, the patient enters medical information into the device. Then, an HCP becomes the current user of the device and the patient remains the device's current patient.

The finding input module 1208 receives and stores findings about patients. In a particular embodiment, the finding input module 1208 is an electronic medical record (EMR) system, which provides a graphical user interface (GUI) for the display and entry of medical information. An example of such an EMR system is the Medcin ChartBuilder(™) GUI. In another embodiment, the finding input module 1208 is an interface for receiving information from a data storage system or computer program that contains findings about a patient.

An HCP engages in various tasks during the treatment or examination of a patient. Tasks include activities, such as: log in, select patient, refill prescription, read correspondence, history of present illness, physical exam, review/update patient medical social and family history, diagnose, select billing code, order tests, order treatments, order laboratory tests, select medications, review literature, recommend consultation, review past charts, annotate past charts, and council patient. Within these tasks there may be subtasks. For example, within “select medication”, a user engages in activities, such as select a category of medication, select a specific medication, review warnings about a selected medication, review drug monographs, select a dosage, select a route, and confirm selected medication.

As a user performs tasks and subtasks, the task select module 1210 stores in the HCP system a state indicating the current activity of the user. In one embodiment, the task select module 1210 detects when a user selects the “read notifications” task. In this embodiment, algorithms relating to a patient are displayed during a designated step in the treatment of that patient. In one embodiment, the task select module 1210 detects each navigation action of a user that is accessing an electronic medical record (EMR) system. In this embodiment, subsets of algorithms relating to a patient are displayed as separate tasks under the EMR system and are selected by the HCP. For example, a message describing the appropriate step therapy of antibiotics for ear infections is displayed in a banner when the HCP selects the prescription-writing task while treating a current patient for which the HCP has entered a diagnostic finding of ear infection. For example, an audible alert is sounded and a banner add describing the appropriate step therapy of antibiotics for ear infections is displayed when the HCP selects an expensive, high-potency antibiotic during the prescription-writing task while treating a current patient for which the HCP has entered a diagnostic finding of ear infection and for which there is no stored finding of recent use of first-line antibiotics.

The algorithm selection module 1216 selects one or more algorithms to display based on one or more factors such as the identity of the current patient, the identity of the current HCP user, the facility with which the HCP system is associated, findings about the current patient, and the current task. It is of note that the algorithm selection module 1216 selects algorithms relating to the current patient in at least two ways. First, the algorithm is selected by selecting algorithms based on the patient identity. For example, an algorithm can include “MATCH PATIENT_ID John Doe” as a selection condition. Second, the algorithm is selected by selecting algorithms based on findings, which are by definition medical data about a particular patient. For example, an algorithm can include “MATCH SMOKING_STATUS Yes” as a selection condition.

In one embodiment, the algorithm selection module specifies selection criteria in terms of the controlled medical vocabulary used by the HCP system to store patient information. In this embodiment, a conversion module converts an algorithm specified by a DMA using a different controlled medical vocabulary into the HCP controlled medical vocabulary.

In a particular embodiment, the algorithm selection module 1216 iterates across all stored algorithms. For each stored algorithm it executes a function that takes as input (a) an algorithm's selection condition that specifies a Boolean equation based on the HCP ID, patient ID, finding values, and task and (b) the current stored values of the HCP system's current HCP user, current patient, findings about the current patient, and current task. The algorithm selection module 1216 instantiates the current stored values into the selection condition's equation and evaluates that equation. This function returns the Boolean value TRUE if the stored values satisfy the selection condition and FALSE otherwise. After executing the function for each stored algorithm, the selection module 1216 returns the set of algorithms for which the function returns TRUE.

In another preferred embodiment, the algorithm selection module 1216 iterates across all stored algorithms. For each stored algorithm, it executes a function that takes as input (a) an algorithm's selection condition that specifies a computer function that returns a numeric value and that takes as argument the HCP ID, patient ID, finding values, and task and (b) the current stored values of the HCP system's current HCP user, current patient, findings about the current patient, and current task. The algorithm selection module 1216 executes the selection condition's function using the current stored values as input. This function returns a numeric value: 0 if the algorithm is irrelevant to the current patient and a positive value if the algorithm is relevant. Higher positive values are regarded as indicating higher importance or relevance. After executing the function for each stored algorithm, the selection module 1216 returns the algorithm for which the function returns the higher numerical value.

Other algorithm selection module embodiments are possible. For example, the efficiency of execution could be improved by storing algorithms in a tree data structure based on the current task and finding values that satisfy each algorithm's selection condition and then traversing the tree based on the current stored values.

In one embodiment, the system enforces access control on algorithms, restricting which patient information they can access, which findings they can read, which findings they can write, which entities they can transmit information to, and which information can be transmitted. In one embodiment, the system enforces that algorithms issued by a particular DMA D only match for patients that DMA is authorized to access. In this embodiment, each patient is associated with one or more DMAs (e.g., the patient's insurance company, HMO, and pharmacy benefits management company.) The system may require an algorithm issued by DMA D to include the condition “MATCH PATIENT_DMA INCLUDES D”. Such an arrangement allows the system to enforce security and privacy rules by preventing unauthorized access to patient medical data.

The algorithm display module 1218 displays medical data associated with an algorithm to the HCP user. Examples of display methods include visual interface, such as displaying a text message, displaying a list of titles of one or more text messages, displaying an icon, displaying a graphical or textual banner, displaying a dialog box, highlighting an item on the screen, changing the order of one or more items on a template for input or output of medical data displayed on a screen, adding one or more items to a template for input or output of medical data displayed on a screen, displaying information, displaying a dialog, displaying a template for input, displaying an approval template, preselecting a displayed element, and displaying an animation. Examples of display methods include audio methods, such as sounding a beep, sounding a series of sounds, or outputting spoken words. Examples of display methods include tactile methods, such as vibrating the HCP device or vibrating an input/output device. Examples of display methods include multi-device methods, such as issuing a telephone call to an HCP portable or non-portable phone, issuing a page to an HCP pager, issuing a telephone call to a clinic, issuing a fax transmission to an HCP or clinic, or sending an email to an HCP or clinic. In one embodiment, a transmission module is associated with the display methods where the transmission module sends some medical finding information to at least one DMA.

In one embodiment, the system displays algorithms using a GUI interface that resembles web-based email. In this embodiment, when an HCP user works with a patient, an icon is displayed if algorithms have been selected as relevant for that patient. When an HCP user enters the view messages task, a list of message titles from the medical data of each selected algorithm is displayed. When an HCP user selects one of the messages and enters the view that message task, the body of the message from the medical data of that algorithm is displayed. In one embodiment, that body is a simple textual message. In another embodiment, that body is an HTML or XML form that can be filled out by the doctor and transmitted to the DMA that originated the message.

In one embodiment, the system displays medical data by issuing a tactile alert, such as by vibrating the HCP device. For example, in this embodiment, a selection condition evaluates to true when the patient is enrolled with pharmacy benefits company B, the current task is select medication, and the current medication selected does not appear on company B's formulary list. When this selection condition evaluates to true, the medical data associated with the algorithm is a directive to issue a tactile alert by vibrating the HCP device.

In another embodiment, the system displays medical data visually by displaying a banner containing the medical information in pictorial form (e.g., GIF or JPEG), animation form (e.g., FLASH or MPEG), or text form (e.g., text, XML, or HTML) in an area on the screen of the HCP device. In this embodiment, when a selection condition evaluates to true, the medical data associated with the algorithm is a directive to display a banner and a reference to a storage location containing that banner. This reference to a storage location containing the banner may refer to local system DRAM memory (e.g., using a memory address), local system disk (e.g., using a file name or database query), or remote system storage (e.g., using a URL or URN). When the selection condition for such an algorithm is triggered, the algorithm display module displays the specified banner on the HCP device screen.

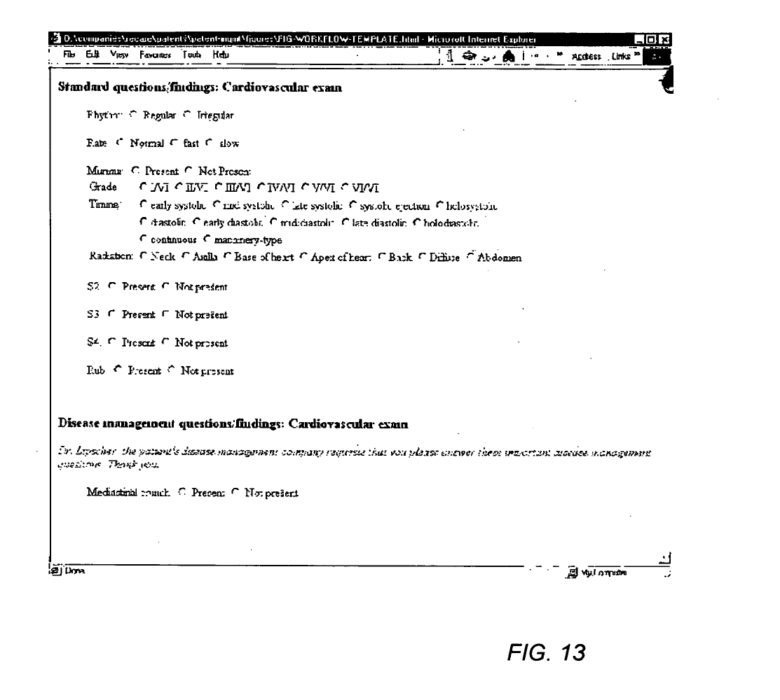

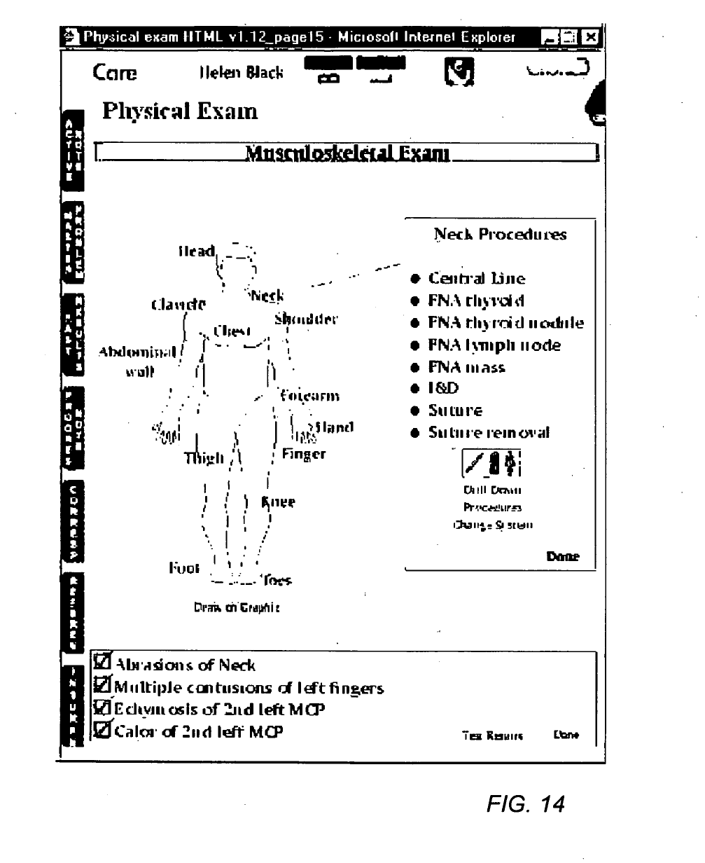

In a further embodiment, the system displays medical data visually by altering a template. A template is a medical device for displaying or gathering medical information. For example, FIGS. 13 and 14 illustrate EMR templates for entering and viewing findings. In this embodiment, the medical data to be displayed include a template ID, a list of new template elements to add, a list of existing elements on that template to delete, and a list of the order in which template elements should be displayed. Rather than display the original default template, the system modifies the default template by applying the transformations described in the medical data to display an altered template. FIG. 13 illustrates the display of an altered template with one question added by the algorithm display module.

In one embodiment, the system provides several display modules including web-based email message display, tactile alert, banner display, and modify template. In this embodiment, the medical data associated with each algorithm includes a DISPLAY_TYPE field and a DISPLAY_CONTENT field. The system displays the content stored in the DISPLAY_CONTENT field using a method specified by the DISPLAY_TYPE field.

The medical data transmission module 1214 of FIG. 12, transmits stored medical data to a DMA system. In one embodiment, stored medical data is sent independent of the algorithms issued by the DMA system. For example, the HCP system regularly sends all findings information stored about DMA D's patients to DMA D's DMA system. In another example, the HCP system regularly sends anonomized information about all patients to a research university. In a further example, the HCP system sends updates about medication and allergy information about patients registered with a particular pharmacy benefits management company C to C's DMA system whenever an update is made to that information.

In one embodiment, algorithms include directives to send specific information to specific DMA systems. For example, when an algorithm is selected by a selection module, the medical data for the algorithm may direct the system to transmit specific information to a specific DMA. For example, an algorithm selected when a particular test is ordered for patients of a particular DMA sends findings that indicate whether that test will be reimbursed to the DMA. In one embodiment, this information is sent automatically without intervention by the HCP user. In another embodiment, this information is sent after being displayed to and updated by the HCP user.

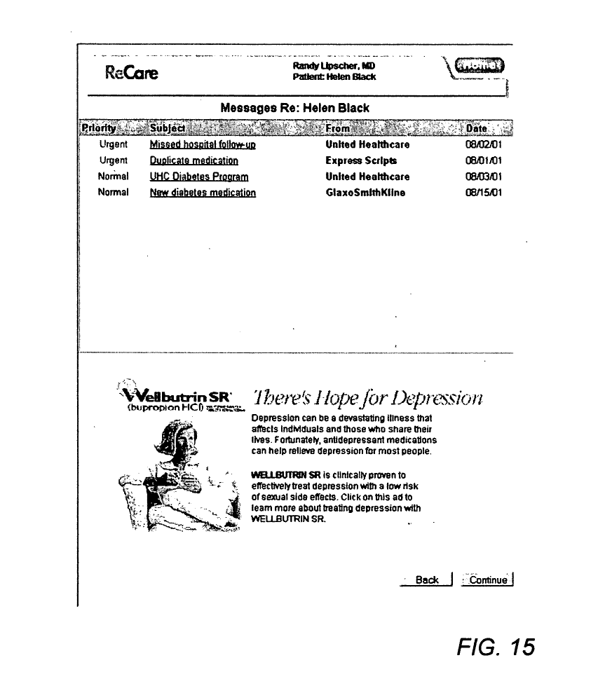

In one embodiment, the HCP system implements an interface similar to that of web-based email. In this embodiment, DMA systems send algorithms to HCP systems. These algorithms specify as conditions for display at least one patient ID or finding condition. The HCP user logs into the system. Then the HCP user repeatedly selects a current patient and examines and treats the current patient. While treating the current patient, the HCP selects the “Display messages” task. When the HCP selects this task, the HCP system selects from the set of stored algorithms those algorithms that either include the current patient ID as their selection condition or that include a set of one or more finding conditions that match findings stored for the current patient. These selected algorithms are displayed as a list, where each item on the list includes a priority, title, and identity of the issuing DMA, which are stored as fields of the medical data associated with the algorithm. FIG. 15 illustrates such a display. The HCP selects one or more messages for more detailed display. Each time a message is selected for more detailed display, the body of the message is displayed. In one embodiment, the body contains text. The HCP has the option to activate a reply button, compose a textual reply, and transmit that textual reply to the DMA specified by the algorithm.

In another embodiment, the message body contains an XML template. For example, the template provides a list of questions that may be answered by inputting medical findings. The answers to these questions are initialized to the findings present for the current patient or left initialized to unanswered if no matching finding has been entered for the current patient. The HCP updates this template by entering findings about the current patient. These findings are stored to the current, patient record. When the HCP has completed updating these findings, the HCP activates a “reply” button on the screen, causing the HCP system to transmit the findings on the template to the DMA.

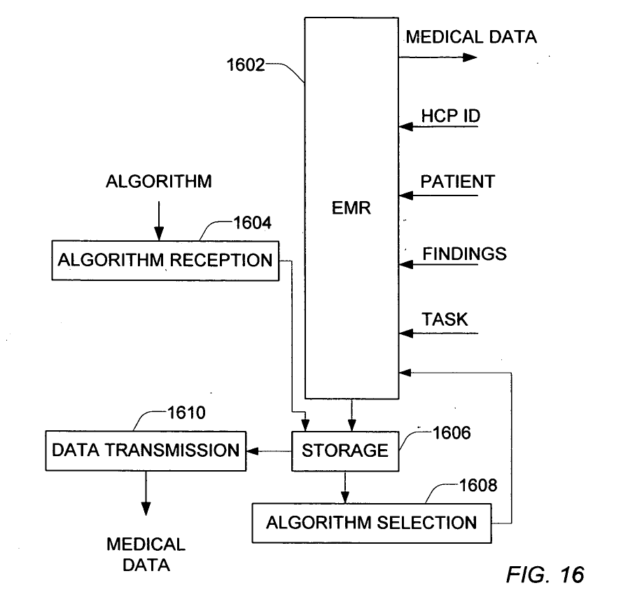

In one embodiment, the HCP system is integrated with an electronic medical record. As illustrated in FIG. 16, the HCP system comprises a reception module 1604, a storage module 1606, a data transmission module 1610, and an EMR module 1602.

The HCP may include a CPU and memory, input and display devices, such as screens, keyboards, mice, etc. permanent storage devices, and a network access. The HCP may communicate using encryption, authentication and privacy algorithms. In one exemplary embodiment, the DMA system only accepts messages from the HCP system. In another exemplary embodiment, the HCP system only accepts messages from the DMA system.

The EMR module 1602 includes interfaces to log in (identify user), select patient, and select task. The EMR module 1602 further includes several tasks interfaces, such as HPI (history of present illness), ROS (review of systems), Dx (enter diagnosis), and Rx (enter prescription). Each of these task interfaces is regarded as a template that includes interface elements for inputting medical findings, displaying medical findings, or both. In this embodiment, as each task is selected, the stored current task state is updated and provided to the selection module 1608 as input. In this embodiment, each of these tasks provides a display interface to the HCP system, which allows algorithms to display medical data during execution of a task. Examples of display methods supported by this display interface include displaying a banner, highlighting a question on a template, adding a question to a template, reordering questions on a template, displaying an icon on a template, and displaying a dialog box.

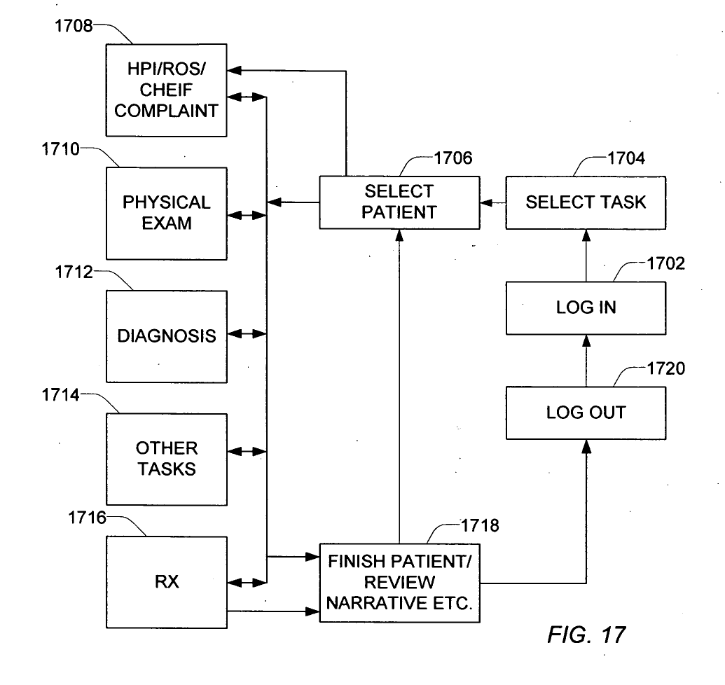

Generally, a physician follows a pattern of tasks. FIG. 17 illustrates an exemplary procedure. For example, a physician may log-in to a portable device and the healthcare provider system, as shown at step 1702. The physician selects a general task, as shown at step 1704. In one exemplary embodiment, the general task includes visiting a patient. The physician may select a patient, as shown at step 1706. The physician may select a specific task relating to the patient visit. Generally, the physician follows an ordered procedure. However, the physician may skip steps or jump to previous steps. In one particular embodiment, some of the steps are performed prior to the physician's patient visit.

In this exemplary embodiment, the physician performs an HPI, ROS, and chief complaint review, as shown at step 1708. The physician may then perform a physical exam, diagnosis, other tasks such as order procedures and tests, write a prescription, and review a note or narrative, as shown in steps 1710, 1712, 1714, 1716 and 1718, respectively. The physician may then log-out, as shown at step 1720.

Each of these tasks may be documented using an interface that includes control elements for indicating findings associated with the step in the procedure. The findings may be recorded in an electronic medical records system based on a controlled medical vocabulary. In addition, the electronic medical record (EMR) system may configure the interface based on disease management algorithms. For example, when the selection conditions of the disease management algorithm match conditions found in the EMR system, the interface may be adjusted based on the disease management algorithm's coding. For example, selection of a patient may activate a message associated with that patient. In another example, selection of a test or procedure order may activate a payer rule that indicates an interest in more data before the order is approved. In a further exemplary embodiment, the selection of a prescription given a set of findings may result in the display of a warning message or the encouragement to prescribe an alternative medication.

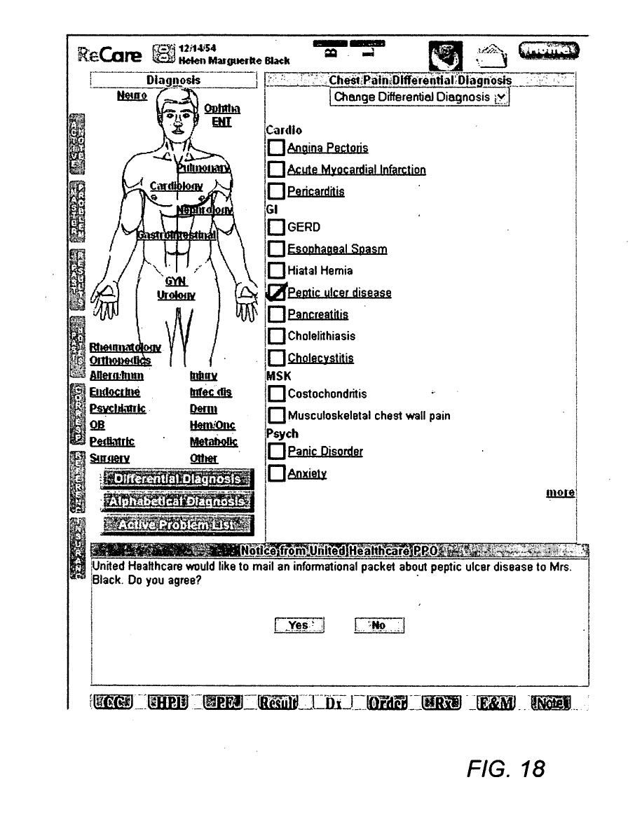

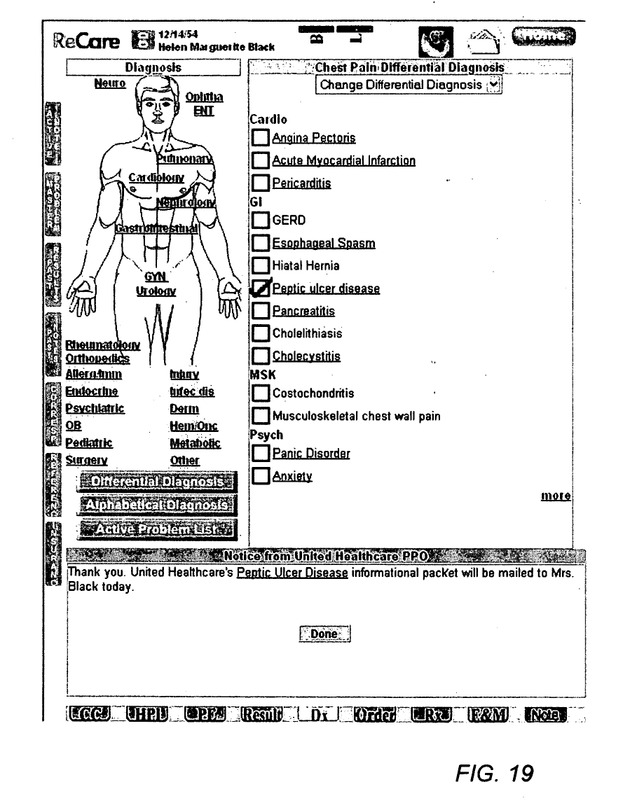

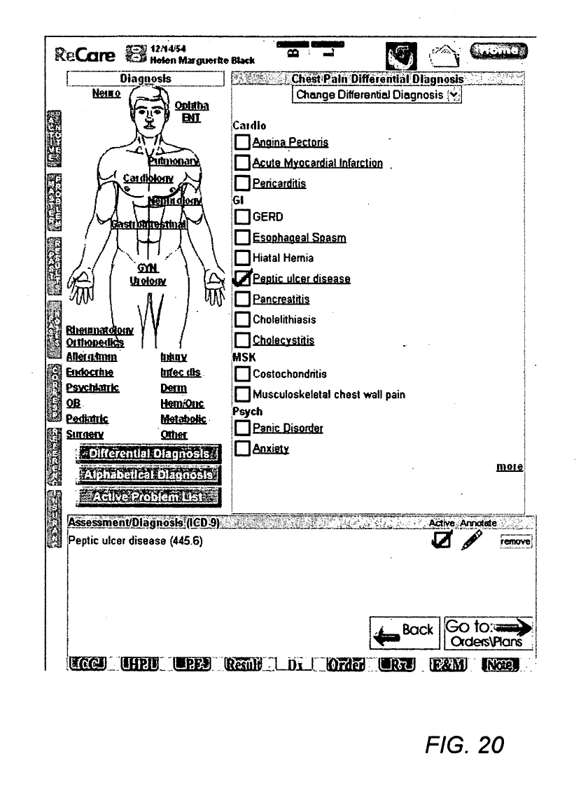

For example, FIG. 18 illustrates an example in which selection of a finding in an EMR system results in a message asking the physician to provide permission to send the patient information about their condition. The physician may, for example, ask the patient whether the patient would like information to be sent and indicate the patient's response using the control elements provided. When, for example, the physician indicates yes, the system may display another message, such as a thank you message or indication that the response was received, as illustrated in FIG. 19. As illustrated in FIG. 20, the physician is then permitted to continue with the patient visit.

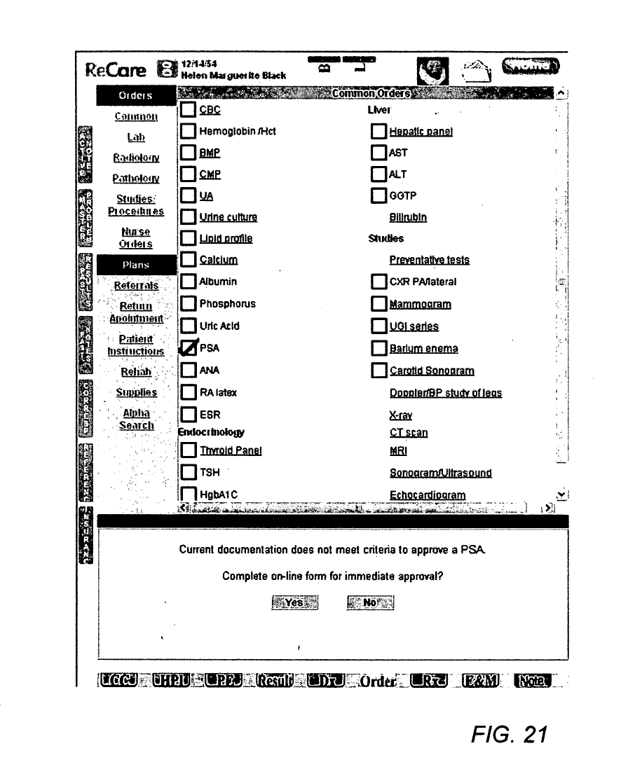

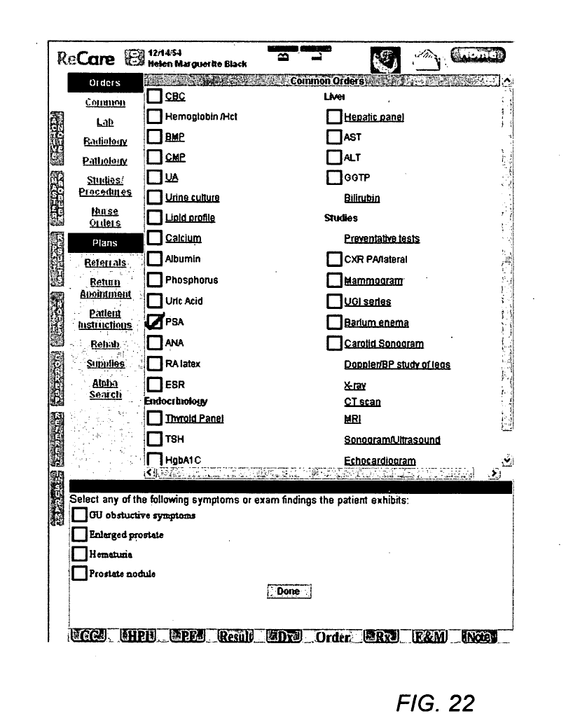

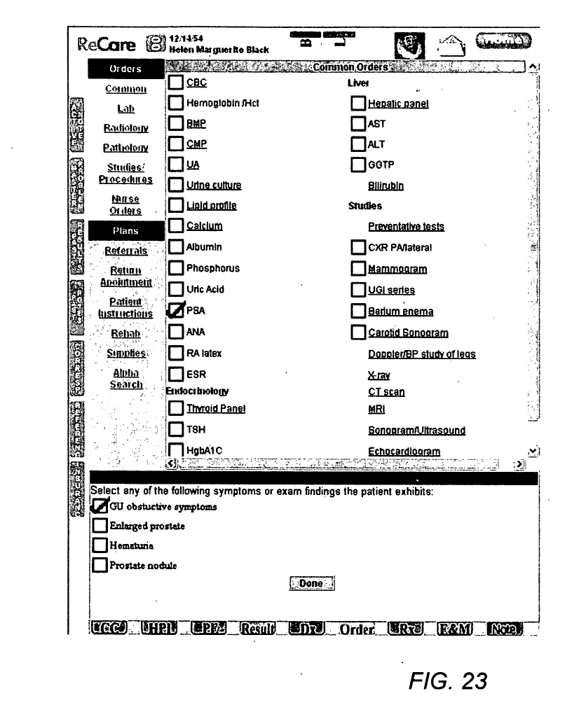

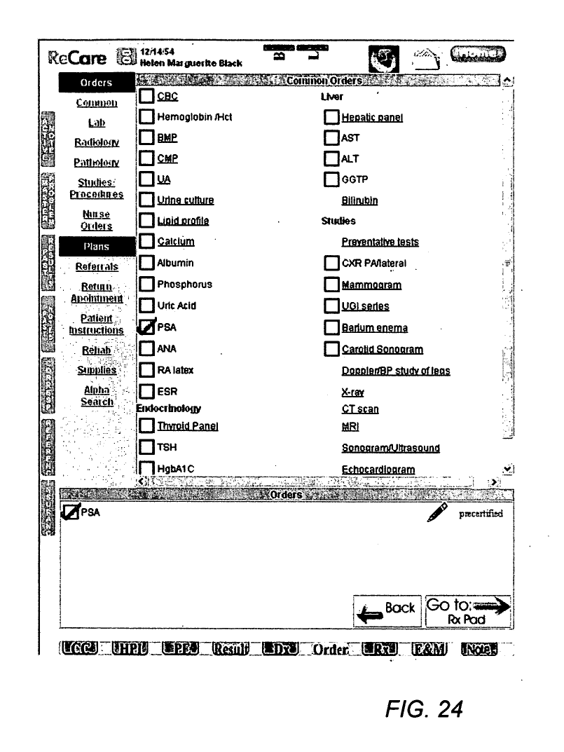

In another exemplary embodiment, when a physician is ordering a test or procedure, a DMA within the system may indicate that the current documentation does not support ordering the test, as illustrated in FIG. 21. The physician may be prompted to enter more information and may be provided with a shortened template for providing the additional information, as illustrated in FIG. 22. The physician may provide additional documentation, as shown in FIG. 23, and proceed in ordering the test, as shown in FIG. 24.

The DMA associated with this procedure may indicate the rule associating the set of findings or lack thereof with the ordered test. This rule may be converted based on the EMR's controlled medical vocabulary and presented in an interface that is specific to the EMR. Other EMR systems may interpret the disease management algorithm based on their specific controlled medical vocabulary and present the results of the rule in their specific interface format. In this manner, a single disease management algorithm may be implemented in multiple types or brands of EMR systems.

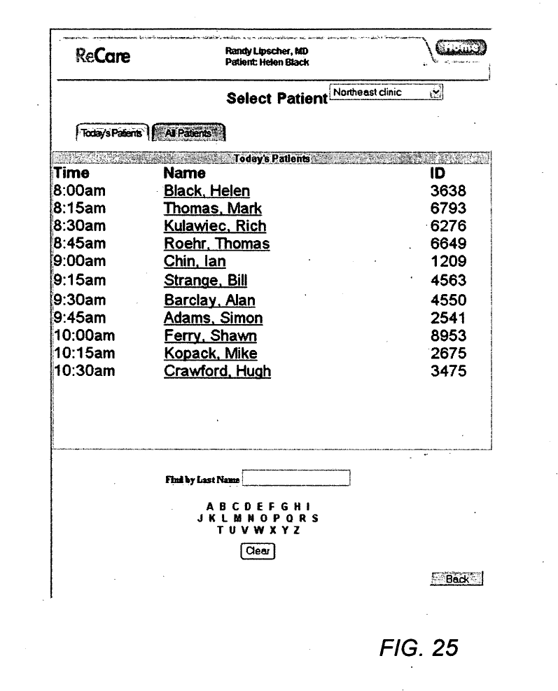

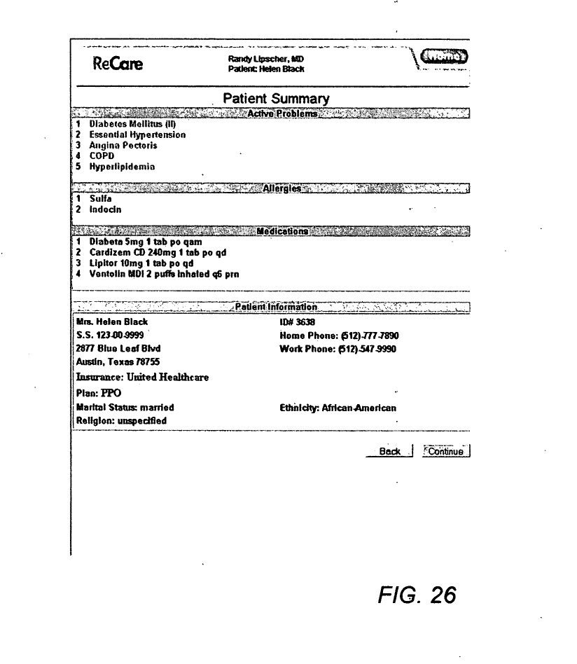

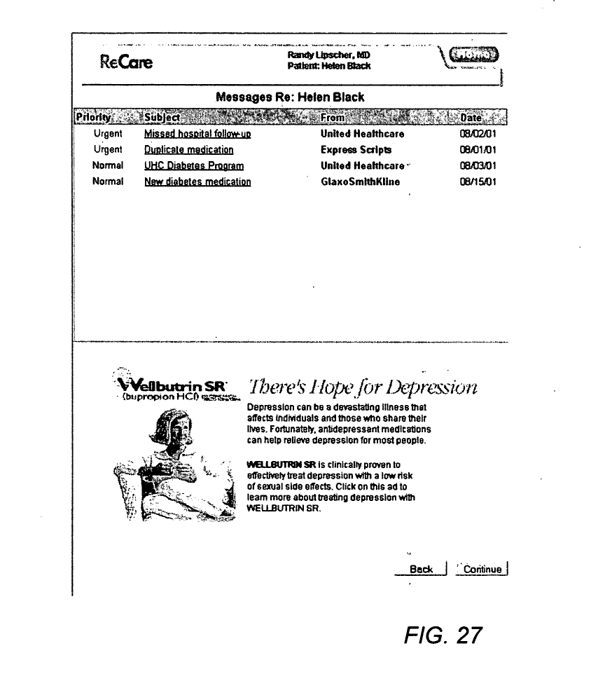

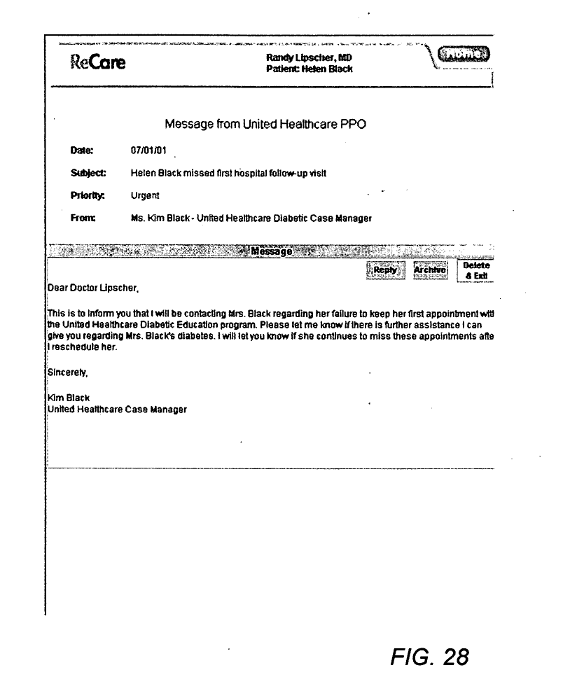

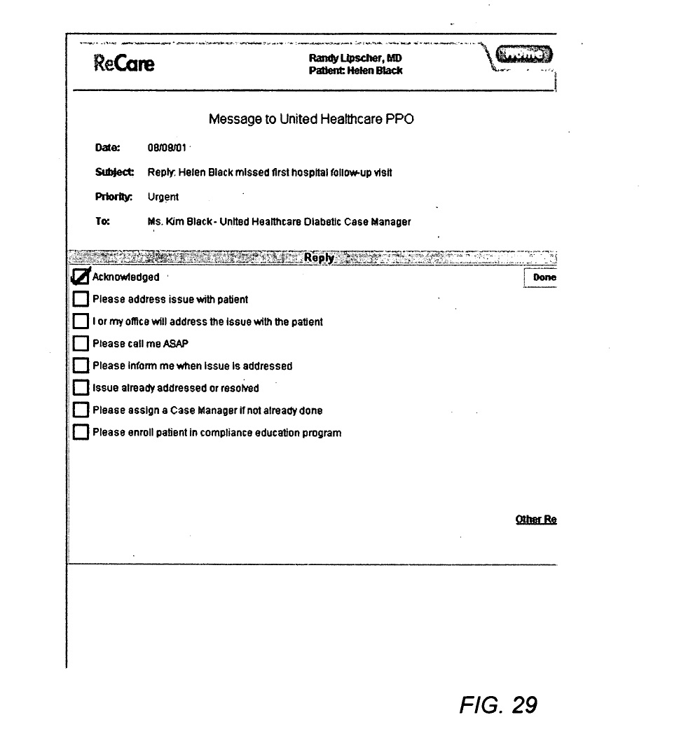

In another exemplary embodiment, a disease management algorithm may be triggered by the selection of a patient. For example, selection of a patient Helen Black, from a list illustrated in FIG. 25 may result in the display of medical data associated with the specific patient and optionally the sending of that data to a DMA advisor, as illustrated in FIG. 26, and may result in access to a set of messages associated with the patient, as illustrated in FIG. 27. The physician may selectively review messages, as illustrated in FIG. 28, and may provide a requested response or reply, as illustrated in FIG. 29.

The DMA system may also provide an interface to the DMA advisor for reviewing the response, sending messages to the physician, and providing data to the physician. In one exemplary embodiment, these messages are encoded in an XML file that includes the addressed HCP system, HCP ID, and patient ID as selection criteria and a message and associated data requests as action items. The XML file may be converted based on the controlled medical vocabulary of the receiving HCP system and implemented in a compatible interface, as illustrated in FIGS. 28 and 29.

In one embodiment, the algorithm identifies patients eligible for a clinical trial. For example, the algorithm includes selection criteria for determining eligibility for a clinical trial. In this embodiment, the algorithm also includes medical data that provides the HCP details about the medical trial, a display module comprising an on-line form for enrolling the patient in the trial, and a data transmission module for transmitting information about enrolled patients to an external DMA, such as the entity conducting the clinical trial.

In another embodiment, the algorithm encourages an HCP user to use approved “step therapy” where inexpensive treatments of tests are attempted first and more expensive treatments or tests attempted only if the inexpensive medications are unsuccessful. For example, the algorithm includes selection criteria for patients of a particular payer DMA, for findings consistent with a particular medical condition, for findings indicating that a less expensive treatment or test has not been recently tried, and for the current task being “select a treatment or test.” In this example, the algorithm also includes medical data formed as a banner alert detailing the recommended step therapy for a medical condition.

In a further embodiment, the algorithm assists an HCP user in following best practices guidelines for diagnosing or treating a condition. For example, the algorithm comprises selection criteria for patients with findings that match specified criteria and for a particular task in an EMR system. In this example, the algorithm also comprises medical data formed as a question added to an EMR template.

In one embodiment, the algorithm assists an HCP in making accurate coding decisions. In this embodiment, the algorithm comprises selection criteria for patients with findings that match specified criteria and for the coding task in a system. In this example, the algorithm also includes medical data formed as a recommended code to be displayed. In another embodiment, the algorithm also includes medical data formed as a set of questions that should be asked to improve coding decisions.

In one embodiment, the algorithm assists a DMA in teaching HCP to make better diagnostic, treatment selection, or coding decisions. In this embodiment, the algorithm includes selection criteria for patients associated with a particular DMA and for the “finish patient” task. In this embodiment, the algorithm also includes a directive to transmit findings from the patient encounter to the DMA system. By using this embodiment, the DMA can track the types of diagnostic, treatment selection, or coding decisions made in different circumstances by a particular HCP and identify cases where best practices data or statistics from other HCPs indicate alternative choices could be considered.

In a further embodiment, the algorithm assists a DMA in teaching HCP to make better diagnostic, treatment selection, or coding decisions. The algorithm includes selection criteria for patients findings that match selection criteria and for the task “view messages”. In this embodiment, the algorithm also includes medical data, such as a recommendation to consider a particular diagnostic decision, treatment selection decision, and coding decision. For example, the medical data can also include a list of findings questions that should be asked to improve medical decisions. For example, the medical data can also comprise references (such as HTML URL links) to articles describing a best practice approach.

In one embodiment, the algorithm provides reference material related to the current patient to an HCP. The algorithm includes selection criteria for patients with findings that match selection criteria and the task “view reference material”. In this embodiment, the algorithm also includes medical data, such as links to articles related to certain findings, such as certain diagnoses, disease processes, systems, medications, and allergies.

In a further embodiment, the algorithm assists DMAs and HCPs in enrolling patients in disease management programs. In this embodiment, the algorithm includes selection criteria for patients belonging to a certain DMA, with findings matching selection criteria, and with findings indicating that they are not currently enrolled in a DMA's disease management program. In this embodiment, the algorithm also includes medical data that assists the HCP in enrolling the patient in the program. For example, in one embodiment the medical data includes a dialog box to be displayed. This dialog box displays text recommending that the patient be enrolled in the program and allows an HCP to select “Yes” or “No”. After the HCP makes a selection, the finding state is updated to indicate this data and the patient ID, findings, and this decision are transmitted to the DMA.

In another embodiment, the algorithm assists DMAs in educating HCPs about particular products. The algorithm includes selection criteria for a task of selecting a product (e.g., selecting a test or medication) and for patients about whom findings match selection criteria. In another embodiment, the algorithm includes selection criteria for a task of selecting a product (e.g., selecting a test or medication) and the subtask of selecting a particular product (e.g., a particular medication). In either case, the embodiment also includes medical data about a particular medical product. For example, for a medication product, the medical data include reference material detailed describing prescripting information (e.g., a drug monograph) for the product. For example, for a medication product, the medical data comprise a banner display indicating conditions when the product should be considered. In another example, for a medication product, the medical data include a banner display offering to send a representative to contact the HCP with more information about the medication.

For a preventive care and immunization based embodiment, in one embodiment, the DMA, HCP, and patients use the system to regularly perform and document preventive care procedures according to the practices recommended by the DMA. For example, a DMA (which is referred to as DMA-A) may wish its male patients over the age of 40 to receive a prostate exam once every year. The DMA could specify this as a treatment algorithm with selection criteria selecting patients (a) associated with the DMA, (b) male, (c) over 40 years old, and (d) who have not had a prostate exam during the past year. The DMA system transmits the treatment algorithm to the healthcare EMR system. Then, when the HCP is accessing the physical exam task, if the current patient meets those criteria, the healthcare EMR system prompts the user to perform the exam (for example, by displaying a banner graphic indicating that a yearly prostate exam is recommended for the current patient). Finally, when the HCP completes the encounter, the healthcare EMR system includes the results of the prostate exam in the report sent to the DMA.

Preventive care prompting may also be driven by specific data elements entered. For example, some DMAs may wish to remind HCPs to perform a yearly foot exam on diabetes patients and to further prompt the HCP to perform such an exam when working with a diabetic patient who has not had a recent foot exam. The DMAs would specify this rule as algorithm selection criteria with medical data to be displayed to prompt the HCP to perform such an exam, and the HCP system would prompt the physician during the physical exam of such a patient.

In another embodiment, a DMA modifies only a particular template. For example, DMA-A can augment the template for “fracture” to, for example, encourage calcium supplements for middle-aged or older females. In this embodiment, the selection conditions include the DMA, the gender and age of a patient, and a diagnostic finding, and the medical data to be displayed include an additional question to be included on the fracture treatment template.

For an exemplary patient education task embodiment, in one embodiment, the DMA, HCP, and patients use the healthcare EMR system to transmit educational material to patients. In another embodiment, a DMA may develop a set of smoking cessation information that they wish to transmit to their smoking patients. The DMA may prompt the HCP to authorize sending that information when the patient visits the HCP when the HCP is engaged in PMFSH task for the patient and to send that information only if the HCP approves.

Similar rules may allow a DMA to communicate diet, exercise, diabetes management, pregnancy, and other information. In one embodiment, a DMA may wish to send educational material to its patients while the patients are using the healthcare EMR system to enter pre-clinical encounter information. For example, in one embodiment, the healthcare EMR system provides Patient-PMFSH review, Patient-HPI, and patient-INFO tasks that allow patients to fill in updates to their past medical, family and social history, to enter the history of their present illness and to view selected health information while they wait in the clinic waiting room. For example, if a patient indicates that they have a fever, the DMA may wish to display rules for distinguishing a cold from a fever to the patient while they wait.

Similar arrangements allow a DMA to provide smoking cessation, diet, exercise, cancer screening, heart disease and similar information to appropriate patients while those patients are in a clinic waiting room or at home filling out pre-clinical information. Such an arrangement would also allow a DMA to inform a patient about general procedures or features of the DMA, such as “Call 1-800-my-nurse for answers to any medical question,” or “Go to http://www.DMA-A.com for answers to your health questions” or “Emergency room visits are only reimbursable if DMA-A is notified within 48 hours of the visit” and so on.

For resource utilization based embodiments, the healthcare EMR system provides methods whereby DMAs can offer guidance to HCPs to improve resource utilization to reduce costs and improve quality of care. For example, the DMA may specify that certain medications, such as those on a “formulary list”, are preferred to others. This can be accomplished, for example, by highlighting on-formulary medications, highlighting off-formulary medications, or adding a “banner” of information for medication X that is displayed when medication Y is displayed.

In another example, the DMA may specify a set of questions that the HCP should answer to get approval to prescribe a given “off-formulary” medication. In one embodiment, this approval process is accomplished by specifying a question with ID OFF-FORMULARY-DMA-1-MEDICATION-X for approval of medication X by DMA-1 that is displayed when medication X is displayed or when medication X is selected. In another embodiment, if the set of questions is long, this approval process is accomplished by specifying one initial question that is displayed when medication X is displayed and specifying several additional questions that are asked if the initial question is answered and matches a specific condition. In another embodiment, this set of questions is specified as a “prerequisite question” as described above. For example, a similar methodology may be used by a DMA to specify a set of questions that the HCP should answer to get approval to perform a procedure or test.

In another exemplary embodiment, a DMA such as an HMO may use the system to communicate with or guide an HCP who is referring a patient to a different HCP or specialist HCP, such as a surgeon or dermatologist. In one such embodiment, a DMA specifies that when an HCP is working on the SPECIALIST_REFERRAL task and the current patient is associated with the DMA, the system should insert content such as a list of DMA-approved specialists or highlights DMA-approved specialists on the default list. In another such embodiment, a DMA specifies that when an HCP is working on the CARDIAC_SPECIALIST_REFERRAL task and the current patient is associated with the DMA, the system inserts content, such as a list of DMA-approved cardiac specialists or highlights DMA-approved cardiac specialists on the default list. In another exemplary embodiment, a DMA specifies that when an HCP is working on the SPECIALIST_REFERRAL task and the current patient is associated with the DMA and under the condition that the current encounter record includes diagnosis of congestive heart disease, the system inserts content, such as a list of DMA-approved cardiac specialists or highlights DMA-approved cardiac specialists on the default list.

In another exemplary embodiment, a DMA such as an HMO or benefits management company may use the system to communicate with or guide an HCP who is ordering a laboratory test or procedure. In one such embodiment, a DMA specifies that when an HCP is working on the ORDER_LABS task and the current patient is associated with the DMA, the system inserts content, such as a list of DMA-approved labs, or highlights DMA-approved labs on the default list. In another such embodiment, a DMA would specify that when an HCP is working on the ORDER_CBC_LABS task and the current patient is associated with the DMA, the system inserts content, such as a list of DMA-approved lab for CBC tests, or highlights DMA-approved labs for CBC tests on the default list.

In another exemplary embodiment, a DMA would specify to the system that it desires information gathered by HCPs during an encounter with patients associated with the DMA. In one such embodiment, a DMA specifies that for patients associated with the DMA, the system inserts in the FINISH_ENCOUNTER task content a prompt for the HCP to transmit all findings from the encounter to that DMA. In one embodiment, this content element includes a text message requesting that the HCP transmit the information, a set of hidden elements corresponding to the patient's ID and all findings by the HCP about the patient for the current encounter, and an activation button where activating the button causes the hidden elements to be transmitted to the DMA.

In another exemplary embodiment, a DMA specifies to the system that it wishes to enroll “high risk” patients, such as diabetics, in specific disease management programs. In one such embodiment, a DMA specifies that for patients associated with the DMA, the system should insert in the FINISH_ENCOUNTER task content including a prompt for the HCP to transmit the patient's name, ID, and diagnosis to that DMA on the condition that the patient is diagnosed with diabetes. In one embodiment, this content element includes a text message requesting that the HCP transmit the information, a set of hidden elements corresponding the patient's ID and diagnosis, and an activation button where activating the button causes the hidden elements to be transmitted to the DMA. In another exemplary embodiment, a DMA, such as a medical research institute, specifies to the system that it wishes to enroll patients meeting criteria specified by the DMA in specific medical studies, such as medication trials. In one such embodiment, a DMA specifies that for all patients, on the condition that the patient's encounter record has fields meeting specified Boolean tests (e.g., age >18 AND gender=F AND smoking=no AND diagnosis=Flu) the system should insert in the FINISH_ENCOUNTER task content including a prompt for the HCP to transmit the patient's name, ID, and diagnosis to that DMA in order to enroll the patient in a study. In one embodiment, this content element includes a text message describing the study and requesting that the HCP transmit the information, a set of hidden elements corresponding the patient's ID, demographic and contact information, and diagnosis, and an activation button where activating the button causes the hidden elements to be transmitted to the DMA.

In another exemplary embodiment, a DMA, such as an HMO or prescription benefits management company, may use the system to communicate with or guide an HCP who is ordering a medication to encourage the physician to select an appropriate medication based on data-driven medicine such as step therapy. In one such embodiment, a DMA would specify that when an HCP is working on the prescription task and the current patient is associated with the DMA, and on the condition that the diagnosis matches a specific diagnosis (e.g., diagnosis=ear infection) and that the patient's medical history does not indicate that a first-line antibiotic has been prescribed for the patient (e.g., medications during past 7 days does not include amoxycillin or tetracycline) and that the current encounter record indicates that a powerful or expensive antibiotic has been prescribed (e.g., new medications includes keflex), the system inserts content such as banner display or message reminding the physician that a specified first-line antibiotic should generally be prescribed before a more powerful or more expensive antibiotic is used (e.g., “For patients with ear infection, the recommended step therapy begins with a 1 week course of amoxycillin or tetracycline. Keflex should generally be prescribed only if the patient does not respond to these initial medications.”)

In further exemplary embodiments, the HCP system may be configured to delete disease management algorithms. For example, a disease management algorithm may have a validity time stamp. The disease management algorithm may be deleted after a period of time or when a similar disease management algorithm having a later time stamp is received. The disease management algorithm may also be configured to direct the deletion of other disease management algorithms. For example, the disease management algorithm may include a specific set of selection criteria or a unique number. An insurance company or a government entity, for example, may periodically update rules and direct that outdated rules be replaced or deleted. In this manner, those only rules that are to be deleted and replaced may be transmitted instead of a complete set of disease management algorithms associated with that entity.

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. A system comprising:
a disease management advisor system configured to receive a disease management algorithm from a disease management advisor;
a healthcare provider system communicatively coupled to the disease management advisor system and configured to receive the disease management algorithm from the disease management advisor system; and
a conversion module accessible to the healthcare provider system and configured to convert the disease management algorithm based on a controlled medical vocabulary.

2. The system of claim 1, wherein the healthcare provider system is configured to provide an interface based on the disease management algorithm.

3. The system of claim 2, wherein the healthcare provider system is configured to receive patient medical information and provide the interface in response to the patient medical information.

4. The system of claim 3, wherein the patient medical information has an associated mapping to the controlled medical vocabulary.

5. The system of claim 3, wherein the patient medical information includes medical findings input.

6. The system of claim 1, wherein the disease management algorithm is coded in an XML format.

7. The system of claim 1, wherein the disease management algorithm includes a set of payer rules.

8. The system of claim 1, wherein the disease management algorithm includes a text message to be displayed based on a medical criteria.

9. A disease management system comprising:
an input interface configured to receive a disease management algorithm;
a selection module configured to select a set of recipients for the disease management algorithm; and
a transmission module configured to transmit the disease management algorithm to a healthcare provider system associated with at least one recipient of the set of recipients.

10. The disease management system of claim 9, wherein the set of recipients includes a physician.

11. The disease management system of claim 10, wherein the physician and the disease management message are associated with a specific patient.

12. The disease management system of claim 9, wherein the disease management algorithm includes a set of rules associated with a specific payer and wherein the set of recipients include physicians eligible to examine patients associated with the specific payer.

13. A healthcare provider system comprising:
an algorithm reception interface for receiving a disease management algorithm;
storage responsive to the algorithm reception interface and configured to store the disease management algorithm; and
a medical data module configured to provide a medical data based on the disease management algorithm.

14. The healthcare provider system of claim 13, wherein the medical data module is configured to transmit patient medical data to a remote disease management advisor system.

15. The healthcare provider system of claim 13, further comprising an electronic medical records module configured to provide a medical data interface and configured to receive patient medical information.

16. The healthcare provider system of claim 15, wherein the patient medical information includes data selected from a group consisting of patient identification, medical findings, healthcare provider identification, and medical workflow task.

17. The healthcare provider system of claim 15, wherein the medical data interface is provided in response to receiving the patient medical information.

18. The healthcare provider system of claim 15, wherein the medical data interface includes an order interface.

19. The healthcare provider system of claim 18, wherein the order interface includes a warning associated with an order violation.

20. The healthcare provider system of claim 15, wherein the medical data interface includes a message associated with a patient.

21. The healthcare provider system of claim 15, wherein the medical data interface includes a prescription interface that includes a warning associated with a formulary violation.

22. A method of disease management, the method comprising:
receiving a disease management algorithm from a disease management advisor;
mapping the disease management algorithm to a controlled medical vocabulary; and
providing a medical data entry interface based on the controlled medical vocabulary, the medical data entry interface configured based on the disease management algorithm.

23. The method of claim 22, further comprising receiving patient medical information via the medical data entry interface.

24. The method of claim 23, wherein the patient medical information includes medical findings data associated with the controlled medical vocabulary.

25. The method of claim 23, wherein the patient medical information includes prescription data.
